(19) United States
(12) Patent Application Publication
(10) Pub. No.: US 2023/0125023 A1
(43) Pub. Date: Apr. 20, 2023

(54) CONCENTRATE CONTAINER ADAPTER AND CONNECTOR HOUSING FOR CONNECTION TO A BLOOD TREATMENT APPARATUS

(71) Applicant: FRESENIUS MEDICAL CARE DEUTSCHLAND GMBH, Bad Homburg (DE)

(72) Inventors: Winfried BREHM, Hofheim (DE); Rafael STERZER, Schweinfurt (DE); Paul SEIT, Schweinfurt (DE); Johannes PLADT, Schweinfurt (DE); Oliver BOND, Heidenfeld (DE)

(73) Assignee: FRESENIUS MEDICAL CARE DEUTSCHLAND GMBH, Bad Homburg (DE)

(21) Appl. No.: 17/908,243

(22) PCT Filed: Feb. 22, 2021
(86) PCT No.: PCT/EP2021/054283

§ 371 Date: Aug. 31, 2022

(30) Foreign Application Priority Data
Mar. 11, 2020 (DE) 10 2020 106 595.7

(51) Int. Cl. A61M 1/16

(52) U.S. Cl. CPC A61M 1/1668; A61M 2205/14

(57) ABSTRACT

The invention relates to a concentrate container adapter for use in a blood treatment device, in particular a dialysis machine. The concentrate container adapter has a fastening body (1) on which a concentrate bag is able to be fastened, wherein the fastening body (1) has a filling opening (11) and two connection openings (12a, 12b) lying laterally on opposite sides of the filling opening (11), a housing receiving region (13) on the fastening body (1), which is arranged in such a way that by means of the housing receiving region (13) the concentrate container adapter is able to be mounted on a connector housing (10), wherein the housing receiving region (13) is arranged externally on the material which is forming the openings.

### TECHNICAL FIELD

The present invention relates to a concentrate container adapter for a blood treatment device, in particular a dialysis machine, and a connector housing for connecting to a blood treatment device, preferably a dialysis machine.

### BACKGROUND

The term “blood treatment device” is understood to mean inter alia a dialysis machine. Dialysis machines are frequently used in dialysis centres for the treatment of a chronic kidney disease.

During the dialysis, the patient's blood flows continuously through the blood chamber of the dialyser, whilst dialysing fluid flows continuously through the dialysing fluid chamber. For the production of dialysing fluid, prefabricated dialysing fluid concentrates can be used, which are diluted with water in the dialysis devices. In dialysis centres, dialysing fluid concentrates are made available either as a prefabricated product in cannisters, bags or cartridges, or are provided from a central tank via a ring line system.

Dialysing fluid concentrates which are made available centrally are easy to handle for the user, but they have the disadvantage that the dialysing fluid can not be coordinated individually to the requirements of the patient. Decentrally provided concentrates indeed permit an individual adaptation of the dialysing fluid to the patient, but they must be brought to the dialysis device for each individual dialysis treatment.

Bags or cartridges which are filled with a pulverulent dialysing fluid concentrate are used in dialysis for the production of a fluid dialysis concentrate. The concentrate bags or cartridges contain a quantity of pulverulent dialysing fluid concentrate which is sufficient for a single dialysis treatment. The bags or cartridges are filled with bicarbonate. Commercially available bicarbonate bags contain 650 to 950 g sodium bicarbonate. Firstly, a fluid bicarbonate concentrate is produced from the pulverulent bicarbonate concentrate. For the production of the dialysing fluid, a further acid concentrate is required, which is provided in a cannister or by a central supply. Bicarbonate concentrate and acid concentrate are then mixed with water to form the finished dialysing fluid.

The dialysis machine therefore has, furthermore, a water connection, via which pure water is supplied. In the dialysis machine, the desired mixing of pure water with the corresponding concentrate subsequently takes place.

The provision of the fluid concentrate via concentrate containers entails various challenges, because the concentrate containers are heavy and therefore laborious for the nursing staff to handle. In addition, for the provision of the concentrate, suction wands of the dialysis machine must be introduced into the concentrate containers, which are inserted into the dialysis machine again after a treatment for cleaning. This handling of the concentrate containers is therefore time-intensive and can lead to a lengthening of the pauses between the individual treatments.

Alternatively, the concentrate can also be fed via a central supply and a corresponding central supply connection on the dialysis machine. However, this presupposes a corresponding equipping of the clinic, and can entail disadvantages with regard to the flexibility of the concentrate which is available and the maintenance effort of the lines which are operated for this.

From EP 1 344 550 A1 a connector is known for connecting a concentrate bag, which is filled with dry concentrate and can be mounted on a dialysis machine. Also in this type of concentrate supply, particular steps are necessary between the treatments. Therefore, a contamination protection arranged on the concentrate bag must be removed manually, the concentrate bag must be inserted, i.e. connected, into the corresponding mount on the dialysis machine and finally connected with the dialysate circuit.

The concentrate bag must be disconnected and removed from the dialysate circuit after a treatment. Only after the removal can a cleaning programme be started on the dialysis machine. The surface cleaning of the dialysis machine can only be carried out after the removal of the concentrate bag by the nursing staff.

In addition to high safety requirements, the time aspect plays a particular role in dialysis treatment, particularly in dialysis centres.

As sufficient time is to be available for the actual treatment, in particular the time between the treatments is subject to a constant time optimization. As described above, between the treatments, in addition to the preparation of the patient, the preparation of the dialysis machine for the next treatment through steps such as the provision of the required concentrate containers or disinfecting of the dialysis machine, takes time.

The present application is therefore based on the problem of providing a cleaning device for a blood treatment device which permits an improved handling and/or a saving of time for the user. In particular, the time required for the preparation of the dialysis machine and the handling steps which are required for this are to be able to be reduced and, at the same time, the safety of the patient is to be guaranteed.

### SUMMARY OF THE INVENTION

The problem forming the basis of the invention is solved by a concentrate container adapter for a blood treatment device according to claim 1, and a connector housing for connecting to a blood treatment device according to claim 6. Advantageous further developments and embodiments are the subject of the dependent claims.

The concentrate container adapter according to the invention, for use in a blood treatment device, in particular a dialysis machine, has a fastening body, on which a concentrate bag is able to be fastened. The fastening body can have a filling opening and two connection openings arranged laterally to the filling opening. Furthermore, the concentrate container adapter can have a housing receiving region on the fastening body, which is arranged in such a way that by means of the housing receiving region the concentrate container adapter is able to be mounted on a connector housing. The housing receiving region can be arranged externally on the material which is forming the openings.

The openings here can be the connection openings and/or the filling opening.

The filling opening and the connection openings can be through-openings and/or can have a circular cross-section. The filling opening can have a larger diameter than the connection openings and can be formed in a cylindrical shape. In particular, the filling opening can have a constant internal diameter. The connection openings can also be partially or completely cylindrical, they can also change in their radius along their course. The centre line of the, preferably cylindrical, filling opening can define a first straight line here. The centre points of the connection openings, which openings are preferably circular in cross-section, can form two points here on a second straight line and define this second straight line.

The centre line of the, preferably cylindrical, connection openings can define a third and fourth straight line. Here, the third and fourth straight line can be formed parallel to one another and/or parallel to the first straight line. The first and/or third and/or fourth straight line can, furthermore, be formed substantially perpendicularly to the second straight line. The connection openings can have here openings lying opposite one another on the third or respectively fourth straight line. The cross-sectional areas of the openings which lie opposite one another can be different from one another here, wherein the opening of the connection openings, which points in the direction of a concentrate bag mounted on the concentrate container adapter, can have a smaller cross-sectional area than the opposite opening. The openings of the through-openings which lie opposite one another, however, can also be spaced apart here perpendicularly to the third, or respectively fourth, straight line. In other words, a fluid flowing through the through-opening can flow in or respectively out at two opposite sides, and is deflected between the two openings within the respective through-opening.

The housing receiving region is arranged externally on the material which forms the openings. In other words, the housing receiving region can be spaced apart from the filling opening and the connection openings. Spaced apart means in this case that the housing receiving opening is not formed in the filling opening and/or in the connection openings. The housing receiving opening here in particular can not lie between the filling opening and the connection openings. In particular, each region of the housing receiving region can lie further away from the centre point of the filling opening and/or at least one of the connection openings, than the length of the connection line between the centre points of the filling opening and at least one of the connection openings. In addition, each point of the housing receiving region can be arranged in such a way that the distance between centre point filling opening and at least the centre point of one of the connection openings is smaller than the distance between housing receiving region and centre point of the filling opening together with the distance between housing receiving region and centre point of a connection opening. Here, the housing receiving region can be formed for example in a subregion around the filling opening. Furthermore, the first, third and fourth straight line can lie on a plane. In addition, the first straight line can intersect the second straight line substantially perpendicularly.

According to a further development of the concentrate container adapter, the receiving region can follow the opening contour of the filling opening. For example, the receiving region can extend in a curved manner around the filling opening and have a first force-receiving area which is formed in an angle of 120 to 60 degrees, preferably 110 to 70 degrees, more preferably 90 degrees, to the first straight line, and can have an edge area which is configured in such a way that the concentrate container adapter is fixed relative to the connector housing.

According to a further development of the concentrate container adapter, the housing receiving region can extend in a frame-like manner over two longitudinal struts on two opposite sides, preferably parallel to the first straight line, of the fastening body away from the main body, and can have at least one first transverse strut, which connect the longitudinal struts.

This transverse strut can serve for force absorption when the concentrate container is coupled to the dialysis device. In a further development, at least two transverse struts can connect the longitudinal struts. The second transverse strut can serve for holding or moving the concentrate container, in particular the first transverse strut can be displaceable into a mounting on the dialysis device by means of manual or automatic moving of the second transverse strut. The first and/or second transverse strut can be formed here in a curved shape. In particular, the second transverse strut can extend in a curved shape between the intersection points of the first transverse strut with the two longitudinal struts. The second transverse strut can be formed here so as to be able to be flapped or folded, so that the second transverse strut is unfolded during carrying by a member of the nursing staff and can be folded up in a position in which the concentrate container adapter is mounted on the connector housing, so that the second transverse strut approaches the connector housing.

According to a further development of the concentrate container adapter, the first transverse strut can have a projection and/or an indent, which is arranged in such a way that it limits a degree of freedom of movement of the concentrate container adapter, when the latter is mounted on a connector housing.

In particular, the projection and/or the indent can prevent a displacing of the concentrate container adapter on the connector housing parallel to the direction of the transverse strut.

According to a further development of the concentrate container adapter, the latter can have a concentrate container detent element on the fastening body, which is arranged in such a way that it limits at least one degree of freedom of movement of the concentrate container adapter, when the latter is mounted on a connector housing.

In particular, the concentrate container detent element can prevent a tilting of the concentrate container adapter around the first transverse strut.

According to the invention, the connector housing for connecting to a blood treatment device has at least one container receiving region, which is formed in such a way that it can receive a concentrate container adapter, preferably according to one of the preceding aspects, on a lower region of the connector housing, and connector connection elements on an underside of the connector housing, which are movable out from the connector housing, and are arranged in such a way that a fluidic connection with the concentrate container adapter is able to be produced.

The lower region of the connector housing means here a region of the connector housing which can not be seen from a top view perpendicularly to an upper side of the connector housing. In other words, the lower region of the connector housing means both an underside, which extends parallel to the upper side of the connector housing, and a face surface which extends from the underside of the connector housing further away from the upper side of the connector housing. The connection of the concentrate bag, which is received in the concentrate container adapter, takes place here from above, i.e. in a direction which extends away from the upper side of the connector housing.

According to a further development of the connector housing, the container receiving region can be a groove-shaped recess on the upper side of the connector housing, which extends on a first straight line.

Here, the groove-shaped recess can be formed so as to be semi-circular in cross-section. The groove-shaped recess can also be formed so as to be rectangular in cross-section with insertion bevels for easy insertion of the concentrate container adapter.

According to a further development of the connector housing, a restriction element can be associated with the groove-shaped recess, which restriction element extends on a second direction, different from the first.

The restriction element can be formed here in the groove-shaped recess. The restriction element can be, for example, a rectangular or a semicircular-spherical elevation in the groove-shaped recess. Here, the restriction element extends in a direction perpendicularly to the upper side of the connector housing, so that a force which acts parallel to the upper side of the connector housing can be received. In other words, the concentrate container adapter can no longer be displaced on an inserting into the groove-shaped recess.

According to a further development of the connector housing, a connector housing detent element can be formed on an underside of the connector housing.

In particular, the connector housing detent element can be arranged on a face surface which extends downwards from the underside of the connector housing, so that with an inserting of the concentrate container adapter, the latter can be prevented by the detent element from a tilting in the groove-shaped recess.

According to a further development, the connector element can have, furthermore, a door element, with a first holding device on a side of the door element, a second holding device for the movable fastening of the door element on the connector housing, wherein the container receiving region for receiving the concentrate container adapter is formed on the door element, wherein the door element is pivotable in such a way about the second holding device so that in a first position the second side lying opposite the first side of the door element is movable under the connector housing, and in a second position the door element extends away from the connector housing.

According to a further development, a wall material of the container receiving region can be formed so as to be flat. Alternatively, the container receiving region can also have a contour. This surface contour can serve as a support for the concentrate container adapter. The housing receiving region of the concentrate container adapter can have a corresponding contour. For example, the contour can be undulating or can have two faces which meet at an angle smaller than 180°. Such a contour can serve to define the position of the adapter more precisely when it is mounted.

The door element can be formed so as to be curved here, and a centre point of the curved shape can lie in the first position of the door element under the connector housing.

According to a further development of the connector housing, a first engagement element can be formed, which is formed on the second side of the door element. A second engagement element, which is formed on the connector housing, can also be formed. Therefore, in the first position of the door element, the first engagement element can engage with the second engagement element. Hereby, a haptic feedback to the nursing staff can take place. In addition, a slight pivoting back of the door can be prevented. Also, further or alternative engagement elements can be provided which engage into one another when the door element reaches the second position.

According to a further development, the connector housing can have a detection unit for detecting a concentrate container adapter.

Here, a portion of the detection unit can be formed on the connector housing and a second portion of the detection unit can be formed ono the concentrate container adapter or on the concentrate bag which interacts with the first portion of the detection unit. The detection unit can be connected here with a control unit, so that a connection of the concentrate container adapter or respectively concentrate bag takes place only when a presence of a concentrate container adapter or respectively of a concentrate bag has been positively detected by the detection unit. In other words, both a presence, the correct position of the concentrate bag and also a presence of the correct concentrate bag can be detected.

According to a further development of the connector housing, the detection unit can be a light transmission unit and a light receiving unit for detecting a presence at a specific position of a reflection geometry.

Here, by means of Snellius' law of refraction it can be precisely determined when the light receiving unit is to detect a reflection. When the concentrate container adapter is inserted in a specific position and it has a region, i.e. a reflection geometry, to which the light transmitting unit is directed, then the change in direction can be calculated on the basis of the material of the region to which the light transmitting unit is directed. In other words, the reflection geometry can only send back a light beam to the light receiving unit when the concentrate container adapter with the reflection geometry is situated in the correct position relative to the connector housing.

According to a further development of the connector housing, the detection unit can be a colour sensor and a light source, which are arranged on the underside of the connector housing.

In this further development, the concentrate container adapter can have a colour coding. The colour sensors can operate here in an interference-based manner and can be, for example, photodiodes with integrated colour filters. Here, also, a control unit is formed for evaluation, so that a connection of the concentrate container adapter only takes place in the case of a positive detection.

According to a further development of the connector housing, the detection unit can be a radar sensor for detecting the movement and position of the concentrate container adapter.

According to a further development of the connector housing, the detection unit can be at least one microswitch.

At a position in which the concentrate bag, or respectively the concentrate container adapter, is situated in a correct position, the connector housing has here at least one, preferably, however, several microswitches.

According to a further development, a receiving device for receiving a concentrate container adapter, preferably according to one of the preceding aspects, has a receiving region with fastening elements for fastening the concentrate container adapter, a mounting region, with mounting elements for mounting the receiving region on a connector housing, preferably according to one of the preceding aspects, a moving region, by means of which the distance between the receiving region and the mounting region is able to be changed in such a way that the receiving region is movable from a region remote from the connector housing to a region close to the connector housing.

According to a further development of the receiving device, the moving region can be formed as a telescope mechanism or as a folding mechanism, and/or the moving region can be moved on a straight line which spans an angle of 20 to 50 degrees, preferably 30 to 40 degrees, between a parallel surface to a base surface in a state in which the receiving device is connected with the connector housing.

According to a further development, a receiving device for receiving a concentrate container adapter, preferably according to one of the preceding aspects, has a mounting region with mounting elements for mounting the receiving region on a connector housing, preferably according to one of the preceding aspects, a receiving region in which the concentrate container adapter is able to be inserted, and a sliding region, which is arranged in such a way, and is inclined to a base surface, in a state in which the receiving region is mounted on the connector housing that a concentrate container is displaceable on a line to the connector housing, wherein the receiving region is formed so as to be pivotable to the sliding region in such a way so that with a pivoting of the receiving region, a concentrate container which is inserted into the receiving region can be displaceable out of the receiving region via the sliding region to the connector housing.

According to a further development of the receiving region, the sliding region can be formed from two track elements running in a parallel manner, and the receiving region can be formed in a curved shape between end sections of the track elements.

According to the invention, a connection system can have a connector housing according to one of the preceding aspects, and a concentrate container adapter according to one of the preceding aspects.

According to a further development of the connection system, this can have a receiving device according to one of claims 18 to 20.

According to the invention, a concentrate container system can have a concentrate container adapter according to one of the preceding aspects, a concentrate container which is able to be filled with a dry concentrate, and/or a concentrate feed line, which can feed a concentrate solution from the concentrate container to a blood treatment device, in particular a dialysis machine.

Through the concentrate container adapter and the connector housing, it is possible to connect the concentrate container adapter with a blood treatment device, in particular a dialysis machine, easily, i.e. with few handling steps. In particular, the concentrate container adapter can be mounted on the blood treatment device, whilst a cleaning step of the connection sockets is still taking place. Through the configuration of the concentrate container adapter, it is possible in addition to prevent a slipping of the concentrate container adapter relative to the blood treatment device, in particular the dialysis machine. Through the concentrate container adapter according to the invention and connector housing, the safety of the patient can be guaranteed.

The features and functions of the present invention described above and further aspects and features are described further below with the aid of a detailed description of preferred embodiments with reference to the enclosed figures. In the figures, the same features/elements and features/elements with the same function are designated by the same reference numbers.

### BRIEF DESCRIPTION OF THE DRAWINGS

There are shown here:

FIG. 1 a concentrate container adapter according to a first embodiment;

FIG. 2 a concentrate container adapter according to a second embodiment;

FIG. 3 a concentrate container adapter according to a third embodiment;

FIG. 4 a connector housing according to a first embodiment;

FIG. 5 a connector housing according to a second embodiment;

FIG. 6 a connector housing according to a third embodiment;

FIG. 7 a schematic diagram of a detection unit according to a first embodiment;

FIG. 8 a schematic diagram of a detection unit according to a second embodiment;

FIG. 9 a schematic diagram of a detection unit according to a third embodiment;

FIG. 10 a schematic diagram of a detection unit according to a fourth embodiment;

FIG. 11 a schematic diagram of a receiving device according to a first embodiment;

FIG. 12 a schematic diagram of a receiving device according to a second embodiment.

### DETAILED DESCRIPTION OF AN EXAMPLE EMBODIMENT

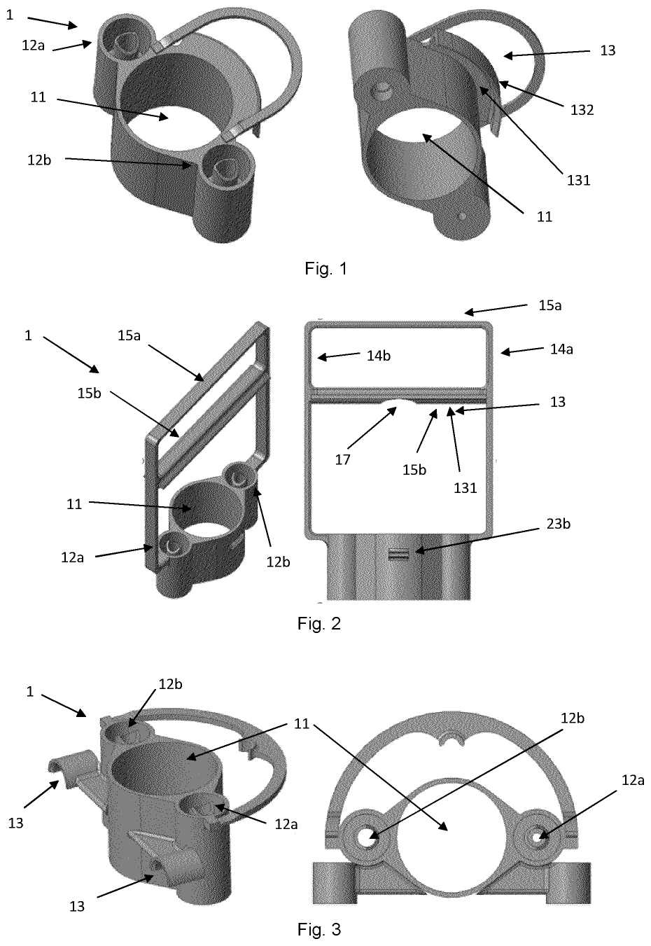

With reference to FIG. 1, a first embodiment will be explained below. Here, FIG. 1 shows a concentrate container adapter according to a first embodiment. The concentrate container adapter has a fastening body 1. On the fastening body 1 a concentrate container, in particular a concentrate bag which is able to be filled with dry concentrate, can be mounted. The concentrate bag can be formed from a flexible material, for example PVC, PVP or polyethylene. The concentrate bag, advantageously still without dry concentrate, can be welded with the concentrate container adapter.

For the preparation of a dialysis treatment, the concentrate container adapter, with a concentrate container mounted thereon, which is now filled with dry concentrate, is fastened to a dialysis machine. In a bicarbonate dialysis, the dry concentrate is bicarbonate concentrate. The fastening body 1 has a filling opening 11. Via this filling opening 11, the dry concentrate can be filled into the concentrate container. The filling opening 11 spans a plane here. The first straight line extends perpendicularly to this plane. The filling opening 11 can be an extending tubular or ring-shaped section, as in the embodiment shown in FIG. 1.

Furthermore, the fastening body 1 has a first connection opening 12a and a second connection opening 12b. These connection openings 12a and 12b span a second straight line. A hose can be mounted respectively at the connection openings 12a and 12b, which hose projects into the concentrate bag. On at least one hose, in particular on the suction end, in addition a filter can be mounted. After filling with a dry concentrate, the filling opening 11 can preferably be closed, for example welded or glued, by a protective foil. Likewise, the first connection opening 12a and the second connection opening 12b are closed by a protective device, for example a protective foil, before a treatment. Before the start of the treatment, more precisely before pure water can be filled from the dialysis machine into the concentrate bag via the concentrate container adapter, the protective device is removed from the connection openings 12a, 12b.

During the treatment, pure water is introduced from the dialysis machine via the first connection opening 12a into the concentrate bag. In the concentrate bag, a mixing of the dry concentrate with the pure water takes place. Subsequently, the concentrate solution, now in fluid form, is directed out via the second connection opening 12b. For this, in the concentrate bag, connected with the second connection opening 12b, a hose can be arranged, which extends from the second connection opening 12b to close to end region of the concentrate bag lying opposite the second? connection opening 12b. The concentrate container adapter, on which a concentrate bag is mounted, is mounted on the dialysis machine before a treatment.

For handling, the concentrate container adapter, as illustrated in FIG. 1, can have a carrying handle, which in this embodiment is preferably formed so as to be foldable. For this, the concentrate container adapter has a housing receiving region 13 on the fastening body 1. Via this housing receiving region 13, the concentrate container adapter is mounted on the dialysis machine in such a way that the concentrate container adapter is no longer movable relative to the dialysis machine. In other words, through this housing receiving region 13 the force transmission is made possible in preferably all degrees of freedom. Via this housing receiving region 13, in particular also the weight force of the concentrate container adapter with concentrate bag is transferred to the dialysis machine, more precisely to a connector housing 10 provided for receiving the concentrate container adapter.

The housing receiving region 13 is formed here around the filling opening 11. The housing receiving region 13 can be formed in a curved shape around the filling opening 11 here, as in the embodiment shown in FIG. 1. A force absorption face 131 extends here away from the first filling opening 11. Via this first force absorption face 131 in particular the weight force of the concentrate bag can be transferred to the connector housing 10. The first force absorption face 131 forms here in the embodiment which is illustrated here an angle of substantially 90 degrees to the first straight line. To prevent the concentrate container adapter being able to shift relative to the connector housing 10, the receiving region has an edge face 132.

The edge face 132 extends from the first force absorption face 131 in a right-angled manner downwards in a direction with respect to a position arranged on the connector housing 10, i.e. contrary to the direction of gravity. As the receiving region is formed in a curved shape, it can also receive forces which act perpendicularly to the curve shape. FIG. 2 shows a further embodiment of the concentrate container adapter. In this embodiment, the housing receiving region 13 extends in a frame-like manner around the filling opening 11. Here, in particular two longitudinal struts 14a, 14b extend away on two sides of the fastening body 1. The longitudinal struts 14a, 14b extend here in particular from two opposite sides of the fastening body 1, more precisely at two sites which lie on the second straight line. The second straight line intersects a centre point of the filling opening 11 here.

Thereby it can be prevented that large tilting moments form. In particular, with a connection of the concentrate bag, i.e. a connecting of the connections on the dialysis side with the first and second connection opening 12a, 12b, a force acts on the concentrate container adapter. At an end region of the longitudinal struts 14a, 14b remote from the main body, two transverse struts 15a, 15b are situated. These transverse struts 15a, 15b are formed parallel here to the second straight line. The transverse struts 15a, 15b are formed spaced apart from one another in such a way that the concentrate container adapter, embracing the first, upper transverse strut 15a, can be easily carried, for example can be formed at a distance of 1.5 to 10 cm, preferably 2 to 8 cm. Via the lower transverse strut 15b, i.e. lying in the direction of the filling opening 11, the force transmission of the weight force onto a connector housing 10 takes place. Here, the connector housing 10, as shown in FIG. 5, has a groove-shaped mount as container receiving region 27b. The connector housing 10 can taper here conically toward the front. In other words, the circumference of the connector housing on a front face, via which the concentrate container adapter must be pushed at the start, can be smaller than at a location at which the concentrate container adapter can finally be inserted into the groove.

The lower transverse strut 15b, as illustrated in FIG. 2b, has an indent 17. Via the indent 17 the concentrate container adapter can be mounted at the correct location on the connector housing 10. In addition, the indent 17 in the lower transverse strut 15b prevents a slipping of the concentrate container adapter relative to the connector housing 10. The concentrate container adapter has furthermore a concentrate container detent element 23a. For this, an extending contour which is semi-circular in cross-section, is formed on a side face of the fastening body 1. The connector housing 10, as illustrated in FIG. 5, has a corresponding counter-contour, which can engage, more precisely latch, with the concentrate container detent element 23a. Hereby, tilting moments of the concentrate container adapter can be received.

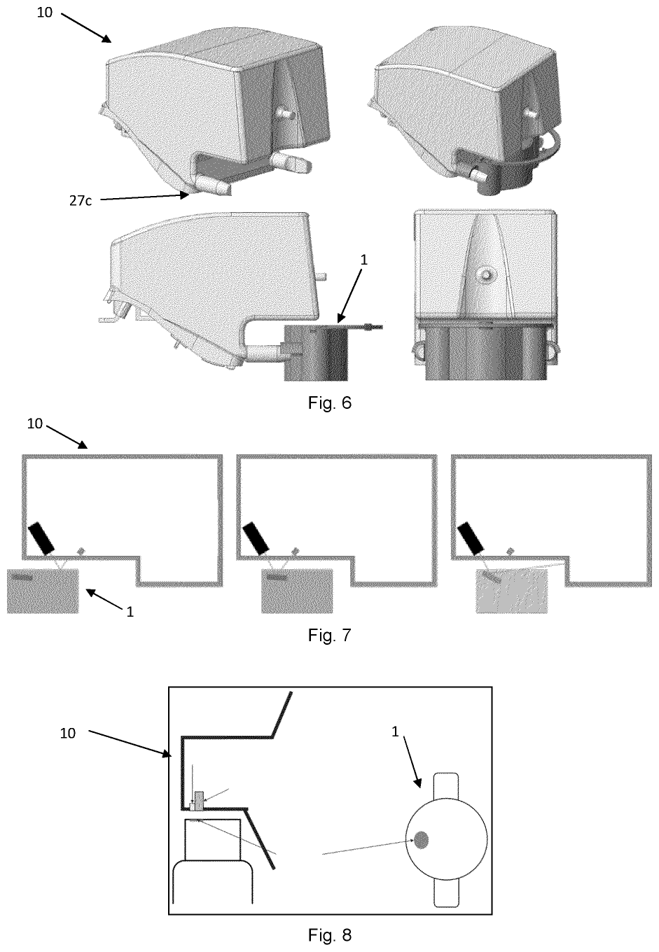

FIG. 3 shows a further embodiment of a concentrate container adapter. Here, the housing receiving region 13 is formed on two sides of the filling opening 11. More precisely, two receiving regions, which are semi-circular in cross-section, are formed, which can shift onto a corresponding counter contour, for instance cylindrical and/or conical pins. The receiving regions therefore project laterally from the concentrate container adapter on a straight line, parallel to the second straight line. A corresponding embodiment of the connector housing 10 is illustrated in FIG. 6. In this embodiment, the pin-like extensions serve as container receiving region 27c. Here, the connector housing 10 can have on its front face an additional, third extension. The concentrate container adapter can be mounted onto this extension. For this, the concentrate container adapter can have a u-shaped recess on its carrying handle.

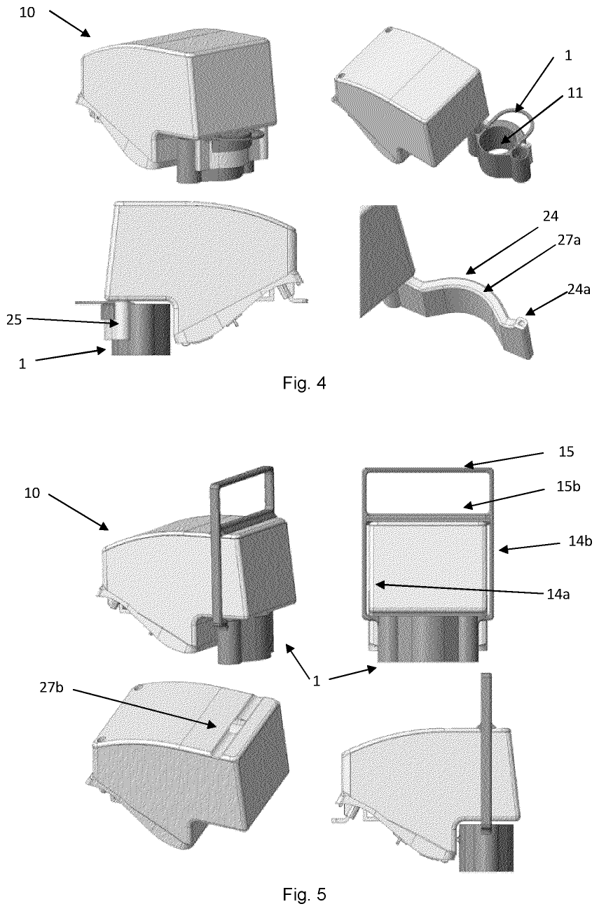

FIG. 4 shows perspective views of a connector housing 10 according to a first embodiment. To receive a concentrate bag, more precisely a concentrate container adapter, the connector housing 10 has a container receiving region 27a. In order to be able to feed pure water to the concentrate bag, and to be able to feed the concentrate solution out from the concentrate bag to the dialysis machine, the connector housing 10 has connection sockets, which are arranged, preferably movably, in the connector housing 10, and can thereby move from above into the mounted concentrate container adapter. Through these connection sockets, thereby a fluidic connection can be produced between concentrate bag and the dialysis machine.

In the embodiment shown in FIG. 4, the connector housing 10 has a door element 24. The door element 24 can be pivoted relative to the connector housing 10. For this, the door element 24 has a cylinder with a bore. The connector housing 10 has, for example, a second cylinder corresponding to the internal diameter of the bore. The door can be placed onto this and subsequently screwed. Alternatively, a hinge joint can connect the door element 24 with the connector housing 10. The container receiving region 27a is formed on the door element 24, wherein a concentrate container adapter of the first embodiment can be mounted via the edge face 132 of the door element 24. The container receiving region 27a, or respectively the door element, can have insertion bevels to facilitate the placing of the concentrate container adapter. Additionally or alternatively, the concentrate container adapter can have insertion bevels. For mounting the concentrate container adapter, the door element 24 is brought into an open position.

In order to be able to receive the concentrate bag, the door element 24 is formed in a curved shape. In closed position, both end regions of the door element 24 are arranged under the connector housing 10 here. In the closed position, a contact face of both end regions of the door element 24 with the connector housing 10 can exist. For this, the end region of the door element 24, which in the open position is not situated under the connector housing 10, can have a first engagement element 24a. The first engagement element 24a, as in the embodiment shown in FIG. 4, can be an indent 17 on the upper side of the door element 24. The connector housing 10 has a corresponding counter contour, for example in the form of a ball element movably mounted by a spring. When the door element 24 is situated under the connector housing 10, the ball element is pressed into the indent 17.

For detecting whether a concentrate bag, or respectively a concentrate container adapter, is situated on the connector housing 10, the connector housing 10 can have a detection unit. For this, as is illustrated in a schematic diagram in FIG. 7, a light source as transmitter, and a light receiver can be formed on the connector housing 10. The transmitter and receiver can be formed here on an underside of the connector housing 10, under which the concentrate container adapter can be received. The concentrate container adapter has on one surface a reflection geometry. When the light source transmits a light beam in a specific direction, the light impulse is received in a corresponding intensity at the light receiver only when the concentrate container adapter is arranged at a predetermined position relative to the connector housing 10.

The reflection geometry on the concentrate bag adapter can vary here, for example depending on the dry concentrate which is situated in the concentrate bag. Thereby it is not only ensured that the concentrate bag adapter is situated at the correct position, rather it is also ensured that the dry concentrate provided for a specific treatment has been prepared. When it is established that an incorrect concentrate container adapter, or this is fulfilled at an incorrect position, in other words, the reflection geometry is not detected by the receiving at the receiving unit, a start of treatment can be prevented. For example, the nursing staff can be presented with a notification, for example in the form of an alarm.

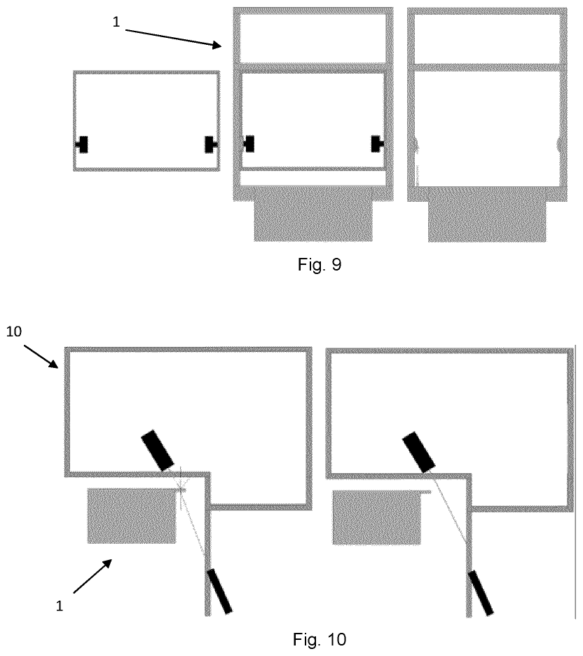

In a further embodiment, the connector housing 10 can have as detection unit a colour sensor as receiver and a light source as transmitter unit. Accordingly, as in the schematic diagram shown in FIG. 8, the concentrate bag or the concentrate container adapter can have a colour marking. Here, according to the colour coding, it can be detected which type of dry concentrate, which concentrate bag type, for example differentiated according to the filling quantity, and a correct positioning can be detected. Also in this embodiment, light source and colour sensor can be formed on an underside of the connector housing 10. As a further embodiment, a radar sensor can be arranged on the connector housing 10, and can detect the position and the presence of the concentrate bag adapter, or respectively concentrate bag.

FIG. 9 shows a schematic diagram of a further embodiment of the detection unit. Also in this embodiment the connector housing 10 has a transmitter unit and receiver unit. The transmitter unit here is a light source. The receiver unit is arranged on the connector housing 10 in such a way that it receives a light impulse only when the correct concentrate bag or respectively concentrate container adapter is situated at the correct position. This is ensured on the basis of the characteristic refractive index of the material which is used. Here, the refractive index of the concentrate bag or concentrate container adapter is taken as the basis for the expected reflection angle. When at a correspondingly positioned receiver unit no previously defined light impulse is received, the previously mentioned conditions of the correct concentrate bag and/or position thereof are not fulfilled. Here, as illustrated in FIG. 9, the receiver unit can be formed on a side of the connector housing 10 lying opposite the underside of the connector housing 10 at an angle.

FIG. 10 shows a further embodiment of the detection unit. Here, the connector housing 10, at a position at which the concentrate bag, or respectively the concentrate container adapter is situated in a correct position, has at least one, but preferably several microswitches. As shown in FIG. 10, the concentrate container adapter can have two projections arranged on the inner sides of the longitudinal struts 14a, 14b. The microswitch on the side faces of the connector housing 10 can be arranged directly behind an elastomer layer, so that with a pressing-in, it is triggered by the projections of the microswitches. The coding of different concentrate bags can take place here via the position of the microswitches on the housing.

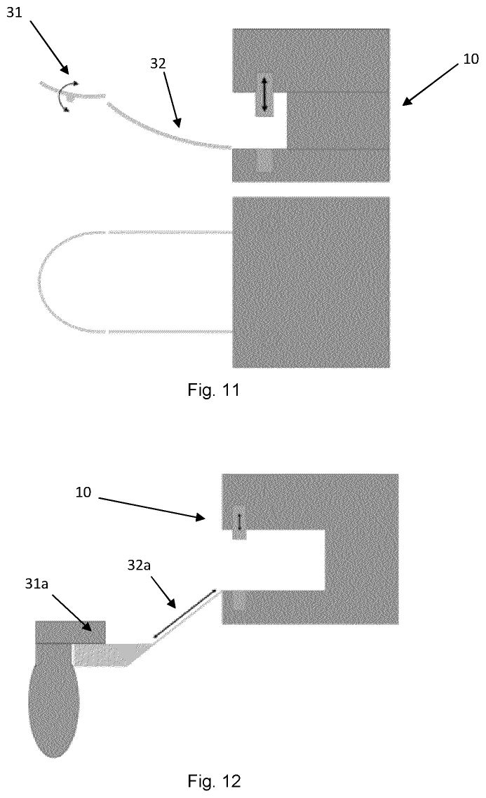

FIG. 11 shows a schematic diagram of a receiving device 3 according to a first embodiment. In the receiving device 3 a concentrate container adapter can be received here, and the receiving device itself can be mounted on the connector housing 10 via a mounting region. In addition, the receiving device 3 has a sliding region 32 and a receiving region 31. Thereby, the receiving region 31, in which a concentrate container adapter is received, can be fed to the connector housing 10 via the sliding region 32. The nursing staff can therefore insert the concentrate container adapter with concentrate bag into a region which is easy to reach. The feeding of the concentrate container adapter itself than takes place via the sliding region 32.

As is illustrated in FIG. 11, the sliding region 32 is formed here as a bar slide, for example as a rigid element. The sliding region 32 can be formed from two track elements running in a parallel manner, and the receiving region 31 can be formed in a curve-shaped manner between end sections of the track elements. Via the rigid element, the concentrate container adapter can be moved or respectively can slip under the connection sockets of the dialysis machine. The receiving region 31 is aligned horizontally as a rotationally movable element at the start, so that the concentrate container adapter remains therein. After completion of the cleaning of the dialysate circuit, the rotationally movable element, the receiving region 31, can tilt and thereby move the concentrate container adapter via the rigid element, the sliding region 32, under the connector housing 10. Thereby, the concentrate bag can be inserted by the nursing staff into the receiving device 3 at any desired time, independently of the progress of cleaning.

FIG. 12 shows a schematic diagram of a receiving device 3a according to a second embodiment. The receiving device 3a has a receiving region 31a with fastening element. In this receiving region 31a the concentrate container adapter can be fastened. Furthermore, the receiving device has a moving region 32a. Via this moving region 32a, the distance between the receiving region 31a and the mounting region can be changed so that the receiving region 31a with the received concentrate container adapter can be fed to the connector housing 10 for connection. At the start, the nursing staff inserts the concentrate container adapter into the receiving region 31a. This can take place independently of the progress of the cleaning. After completion of the cleaning, the moving region 32a moves the concentrate container adapter to the connector housing 10, for connection under the connection sockets. The moving region 32a can be formed as a telescope mechanism or as a folding mechanism.

1. A concentrate container adapter for a blood treatment device comprising,
a fastening body on which a concentrate bag is able to be fastened, wherein the fastening body has a filling opening and two connection openings arranged laterally to the filling opening,
a housing receiving region on the fastening body, which is arranged in such a way that by means of the housing receiving region the concentrate container adapter is able to be mounted on a connector housing,
wherein the housing receiving region is arranged externally on the material forming the filling opening and/or connection openings.

2. The concentrate container adapter according to claim 1,
wherein the housing receiving region follows an opening contour of the filling opening.

3. The concentrate container adapter according to claim 1,
wherein the housing receiving region extends in a frame-like manner via two longitudinal struts on two opposite sides of the fastening body away from the latter, and has at least one transverse strut which connects the longitudinal struts.

4. The concentrate container adapter according to claim 3,
wherein the at least one transverse strut has a projection and/or an indent, which is arranged in such a way that it limits a degree of freedom of movement of the concentrate container adapter when the latter is mounted on a connector housing.

5. The concentrate container adapter according to claim 1, further having
a concentrate container detent element on the fastening body, which is arranged in such a way that it limits at least a degree of freedom of movement of the concentrate container adapter when the latter is mounted on a connector housing.

6. A connector housing for connecting to a blood treatment device comprising,
at least one container receiving region, which is formed in such a way that it can receive the concentrate container adapter of claim 1, on a lower region of the connector housing, and
connector connection elements on an underside of the connector housing, which are movable out from the connector housing, and are arranged in such a way that a fluidic connection with the concentrate container adapter is able to be produced.

7. The connector housing according to claim 6, wherein
the container receiving region is a groove-shaped recess on the upper side of the connector housing.

8. The connector housing according to claim 7, wherein
a restriction element is associated with the groove-shaped recess, which restriction element extends away from and/or to the upper side of the connector housing.

9. The connector housing according claim 6, wherein
a connector housing detent element is formed on an underside of the connector housing.

10. The connector housing according to claim 6, further having
a door element, with a first holding device on a side of the door element,
a second holding device for the movable fastening of the door element on the connector housing,
wherein the container receiving region for receiving the concentrate container adapter is formed on the door element, wherein the door element is pivotable about the second holding device in such a way so that in a first position the second side, lying opposite the first side of the door element, is movable under the connector housing, and in a second position the door element extends away from the connector housing.

11. The connector housing according to claim 6, wherein
a wall material of the container receiving region is formed so as to be flat, or the container receiving region has a contour which serves for the support of the concentrate container adapter.

12. The connector housing according to claim 10, further having
a first engagement element, which is formed on the second side of the door element,
a second engagement element, which is formed on the connector housing, so that in the first position of the door element the first engagement element can engage with the second engagement element.

13. The connector housing according to claim 6, further having
a detection unit for detecting a concentrate container adapter.

14. The connector housing according to claim 13, wherein
the detection unit is a radar sensor for detecting the movement and/or position of the concentrate container adapter, and/or the detection unit is at least one microswitch, and/or the detection unit is a colour sensor and a light source, which is arranged on the underside of the connector housing, and/or the detection unit is a light transmitting unit and a light receiving unit for detecting a presence of a reflection geometry at a specific position.

15. A connection system comprising,
a connector housing comprising at least one container receiving region, which is formed in such a way that it can receive the concentrate container adapter of claim 1 on a lower region of the connector housing, and connector connection elements on an underside of the connector housing, which are movable out from the connector housing, and are arranged in such a way that a fluidic connection with the concentrate container adapter is able to be produced, and
concentrate container adapter.
